(19) United States
(12) Patent Application Publication
(10) Pub. No.: US 2014/0206926 A1
(43) Pub. Date: Jul. 24, 2014

(54) METHODS FOR OPTIMIZING AND EVALUATING DOSE DISTRIBUTIONS IN BRACHYTHERPAY

(71) Applicant: Robert van der LAARSE, Zeist (NL)

(72) Inventor: Robert van der LAARSE, Zeist (NL)

(21) Appl. No.: 13/745,153

(22) Filed: Jan. 18, 2013

(51) Int. Cl. A61N 5/10

(52) U.S. Cl. CPC A61N 5/1031; A61N 5/1007; A61N 5/1039; A61N 5/1064
USPC 600/8

(57) ABSTRACT

Methods and systems are disclosed for radiation therapy treatment planning. In one embodiment, a radiation therapy treatment planning method is disclosed for adjusting a dose distribution of a target anatomical portion. The method includes displaying an image of an implanted volume and a set of catheters displayed as implanted in the anatomical portion. The method also includes determining to adjust the dose distribution to the implanted volume for delivering via the catheters. The method also includes receiving, at a processor, a selection of a dose distribution parameter affecting the strength of the dose distribution and adjusting, based on the received parameter, a dwell weight of the radiation dose at each dose position in the set of catheters, wherein the adjusted dwell weight adjusts the dose distribution relative to a center region of the implanted volume for moving the distribution to or from an outer circumference of the target anatomical portion.

### TECHNICAL FIELD

The present disclosure relates generally to methods for brachytherapy treatment. More particularly, and without limitation, the present disclosure relates to methods for optimizing the dose distribution in brachytherapy treatment.

### BACKGROUND

Brachytherapy treatment systems may treat a patient by inserting hollow treatment catheters (aka “implants”) into a patient's cancerous tissue, such as the male prostate gland, the female breast, or other accessible areas in which a tumor is located. The treatment catheters are connected outside the patient's body with a so-called after-loading apparatus having a radiation delivery unit for advancing one or more energy emitting sources through the catheters, which deliver doses of radiation to the patient's cancerous tissue. In one implementation, e.g., in high dose rate (HDR) brachytherapy, the source may have an active length of about 3.5 mm and may be displaced in a stepwise fashion along a catheter with steps of about 5 mm for effectuating a pre-defined dose distribution around the catheter. Each source may be stopped at pre-defined positions within the catheter (and thus inside the treatment site) for pre-defined times.

In general, the pre-defined positions are known as dwell positions, and the pre-defined times at which the source is halted in a specific dwell position are known as dwell times. Dwell positions and dwell times may be calculated in a treatment planning unit by discrete optimization algorithms. A dwell step may be selected based on a compromise between the dimensions of the volume with cancerous tissues and the source active length. In exemplary embodiments, the dwell step is independent of the anisotropy of the source.

However, treatment planning solutions containing a set of dwell positions and dwell times in the inserted catheters provide a discrete treatment solution. As an energy emitting source is stopped for a certain dwell time at each dwell position, the radiation dose delivered in that position exhibits a distribution having a peak (or height) determined by the length of the dwell time interval at the dwell position as well as other factors, such as the level of activity of the source.

A discrete treatment planning solution of this type may not provide the ideal treatment planning solution when it is intended that the target anatomical portion (e.g., the cancerous organ) should receive a homogeneous dose coverage and that the dose should steeply fall-off directly outside the target anatomical portion, sparing the organs at risk around or near the target anatomical portion.

The use of a single step size for all catheters also restricts the planning software. For example, the position of the source stepping through the catheters within the anatomical portion is limited to these fixed dwell positions. This restricts the placement of the outer dwell position in each catheter in relation to the boundary of the target anatomical portion, leading sometimes to a step size of 2.5 mm (or even 1.0 mm) instead of 5 mm for better dose coverage of the target anatomical portion.

Once the catheters are placed in the patient, the above type of brachytherapy system offers two degrees of freedom: the dwell position and the dwell time. Usually all dwell positions located in the sections of the catheters, that are inside the target anatomical portion, are activated. Then, optimization of the dose distribution may be performed by manipulation of the dwell times, either by the user or by dedicated software.

Most optimization procedures do not determine the absolute dwell times for each dwell position. Instead, they result in a set of relative values for the dwell times (e.g., “dwell weights”) in the range 0 to 1. These relative dwell weights correspond to relative dose values in a set of predefined points, within and around the target anatomical portion and the volumes of organs at risk. The absolute dwell times may be calculated from the stated mean dose in dose points in a given set of points, e.g., the prescription dose (PD). Such a set of points may be defined and located on the target anatomical portion and the organs at risk.

Several factors influence a mathematically optimized dose distribution. Such a dose distribution does not always represent the best possible one in and around an organ that has needles or catheters implanted into it for radiation treatment, as not all clinical aspects of a dose distribution can be translated into mathematical optimization parameters. Thus, different types of optimization algorithms with varying user-defined constraints may all deliver different dose distributions. Therefore, clinical experience may be needed to judge the mathematically optimized dose distribution for each patient treatment. Based on that judgment, changes can be made to the optimization constraints, resulting in a new dose distribution. Several iterations may be required before the final dose distribution will be accepted for clinical use.

In clinical practice, this latter step of fine tuning of the dose distribution is generally a rather time-consuming task and is very patient-specific. Furthermore, it is often unclear and might be even subjective why one plan is preferred in favor of another. Lack of objective criteria goes along with lack of uniform reporting of dose distributions for clinical trials.

Therefore, there exists a need for the development of tools to facilitate and speed up this process and provide objective criteria for evaluating a dose distribution.

### SUMMARY

The present disclosure provides improved systems and methods for providing (post-)optimization of a dose distribution with objective analysis tools for deriving scoring parameters from a dose distribution treatment plan.

In one exemplary disclosed embodiment, a radiation therapy treatment planning method is provided for adjusting a dose distribution of a target anatomical portion having an implanted volume. According to the method, the method may display an image of the implanted volume and a set of catheters displayed as implanted in the anatomical portion. The method may also determine to adjust the dose distribution to the implanted volume that is configured to be delivered via the set of catheters. The method may receive, at a processor, a selection of a dose distribution parameter that affects the strength of the dose distribution to the implanted volume. The method may also adjust, based on the received dose distribution parameter, a dwell weight of the radiation dose at each dose position in the set of catheters, wherein the adjusted dwell weight adjusts the dose distribution relative to a center region of the implanted volume such that the distribution moves to or from an outer circumference of the target anatomical portion.

In another exemplary disclosed embodiment, a radiation therapy treatment planning method is provided for adjusting a dose distribution of a target anatomical portion and an organ at risk, the target anatomical portion being proximate to the organ at risk, the dose distribution including a set of dose points randomly displaced throughout the target anatomical portion. The method may display a first image of the target anatomical portion, the first image including the organ at risk and a set of catheters. The method may also determine that the dose distribution affecting the organ at risk needs further adjustment. The method may receive, at a processor, a selection of a property of the dose distribution. The method may also iteratively adjust, by the processor, based on the received selection, the dose at each point in the set of points until a predefined characteristic of the dose distribution has been achieved, and generate a second image of the target anatomical portion and the organ at risk.

In yet another exemplary disclosed embodiment, a radiation therapy treatment planning method is provided for determining contiguous volumes within a region of interest of a target anatomical portion, wherein a contiguous volume reflects a volume of the anatomical portion having a radiation dose higher than a set radiation dose value. The method may randomly select a first point within a 3-dimensional representation of the target anatomical portion, the first point associated with a higher radiation dose than the set radiation dose value. The method may also iteratively add a set of surrounding layers of points to the first point selected, each layer of points being in a layer around the previous surrounding layer of points and having higher radiation doses than the set radiation dose value. The method may also determine that no points around the outermost surrounding layer to the first point have higher radiation doses than the set radiation dose value. The method may determine, based on the set of points in the set of surrounding layers, a first contiguous volume within the target anatomical portion, wherein the first contiguous volume has a radiation dose higher than the set radiation dose value.

In a further exemplary disclosed embodiment, a computer-implemented method of radiation therapy treatment planning is provided for determining at least the contiguous volumes inside the target anatomical portion, having a radiation dose higher than a set radiation dose value. The method may randomly select a first point within a three-dimensional representation of the target anatomical portion, the first point associated with a higher radiation dose than the set radiation dose value. The method may also iteratively add a set of surrounding layers of points to the selected first point, each layer of points being in a layer around the previous surrounding layer of points and having higher radiation doses than the set radiation dose value. The method may determine that no points around the outermost surrounding layer to the first point have higher radiation doses than the set radiation dose value, and determine, based on the set of points in the set of surrounding layers, a first contiguous volume within the target anatomical portion, wherein the first contiguous volume has a radiation dose higher than the set radiation dose value.

### BRIEF DESCRIPTION OF THE DRAWINGS

The accompanying drawings, which are incorporated in and constitute a part of this specification, illustrate several embodiments of the present disclosure and together with the description, serve to explain the principles of the disclosed embodiments.

FIG. 1 depicts an exemplary ultrasound needle placement system according to the disclosed embodiments, where catheters are inserted into the target anatomical portion under guidance of ultrasound imaging;

FIGS. 2a and 2b depict exemplary isodose distributions of a prostate implant prior to optimization, showing a high dose to the rectum; where FIG. 2a shows the dose distribution obtained when all dwell weights are 1; and where FIG. 2b shows the dose distribution obtained with the Geometric Optimization module, as in known in the prior art;

FIG. 3a depicts an exemplary user interface tool for implementing an enhanced geometric optimization algorithm, according to the disclosed embodiments;

FIG. 3b depicts an exemplary isodose distribution generated by using enhanced geometric optimization, with the settings given in FIG. 3a, according to the disclosed embodiments, where the dose to the rectum is reduced further but where the area displayed above the urethra now has a dose below the prescription dose;

FIG. 4 depicts another exemplary isodose distribution generated by using an enhanced geometric optimization (EGO) algorithm, according to the disclosed embodiments; where FIG. 4 demonstrates that the dose in the area above the urethra is no longer under-dosed as the 100% prescription isodose line runs around the target anatomical portion, where FIG. 4 also displays that the dose to the rectum has increased, such that the 100% prescription isodose line now touches the contour of the rectum;

FIG. 5a depicts an exemplary user interface tool for implementing an interactive inverse planning (IIP) algorithm, according to the disclosed embodiments, where FIG. 5a displays the lower and upper limits after EGO has finished but before IIP starts;

FIGS. 5b and 5c each depicts an exemplary isodose distribution based on the dose limit settings in FIG. 5a, before using IIP, according to the disclosed embodiments; where FIG. 5b depicts a slice at the level of the bladder at the top of the implanted volume, and where FIG. 5c depicts a slice through the center of the implant, showing the rectum being close to the target anatomical portion;

FIG. 6a depicts an exemplary user interface tool for implementing IIP by setting upper limits, according to the disclosed embodiments, when all upper limits set for the organs at risk are met, where FIG. 6a shows the upper and lower dose settings generated by IIP;

FIGS. 6b and 6c each depicts an exemplary isodose distribution generated by the IIP settings in FIG. 6a by setting upper limits for the organs at risk and the lower limit for the target anatomical portion, according to the disclosed embodiments;

FIG. 7a depicts an exemplary user interface tool for implementing IIP by setting a lower limit to the target dose, according to the disclosed embodiments when the required upper limit for the urethra and the bladder cannot be met;

FIGS. 7b and 7c each depicts an exemplary isodose distribution generated by the IIP settings of FIG. 7a by setting a lower limit to the target dose, according to the disclosed embodiments; where FIGS. 7b and 7c demonstrate that the upper dose to the urethra, set to 120% of the prescription dose, is met at the cost of the dose to the bladder, in which the highest dose is 173%, instead of the required 100%, due to the touching of the bladder to the target anatomical portion, keeping the 120% upper limit to the urethra;

FIG. 8 illustrates an exemplary display of dose-volume histogram parameters of a dose distribution, according to disclosed embodiments;

FIGS. 9a-c each illustrates another exemplary isodose distribution generated by enhanced geographic optimization and interactive inverse planning of a prostate implant, according to the disclosed embodiments;

FIG. 10 illustrates an exemplary process for determining contiguous volumes inside an anatomical portion, according to the disclosed embodiments, e.g., by using randomly placed dose points in the implanted volume (e.g., including target organs and organs at risk) to generate a dose volume histogram;

FIG. 11 illustrates an exemplary table reporting the contiguous volumes for given dose limits for an anatomical portion, according to the disclosed embodiments;

FIG. 12 illustrates an exemplary process for implementing an algorithm for searching the optimal settings for enhanced geographic optimization, according to the disclosed embodiments;

FIG. 13 illustrates an exemplary process for implementing interactive inverse planning, according to the disclosed embodiments; and

FIG. 14a-b each illustrate an exemplary process for implementing interactive inverse planning, according to the disclosed embodiments.

### DETAILED DESCRIPTION OF THE EMBODIMENTS

Reference will now be made in detail to exemplary embodiments, examples of which are illustrated in the accompanying drawings. Wherever possible, the same reference numbers will be used throughout the drawings to refer to the same or like parts.

Systems consistent with the disclosed embodiments may involve two stages: (i) a real-time interactive tool (Enhanced Geometrical Optimization or “EGO”) to homogenize the dose distribution over the target anatomical volume, and (ii) a real-time interactive tool (Interactive Inverse Planning or “IIP”) to adapt this dose distribution to the dose constraints in the surrounding organs at risk while keeping the target anatomical portion covered as well as possible. The resulting dose distributions of the EGO and IIP tools may be evaluated by studying a display of upper and lower percentage dose limits obtained in target anatomical portion and any vulnerable regions around it. Also, the dose volumes (e.g., in cm³) corresponding to these dose limits may be displayed. Additionally, to detect clinically unfavorable, large contiguous volumes with a dose much higher than the prescription dose, a real-time tool may be used to determine contiguous volumes with such a high dose limit, based on the same randomly placed dose points on which the dose-volume histograms (DVHs) are based.

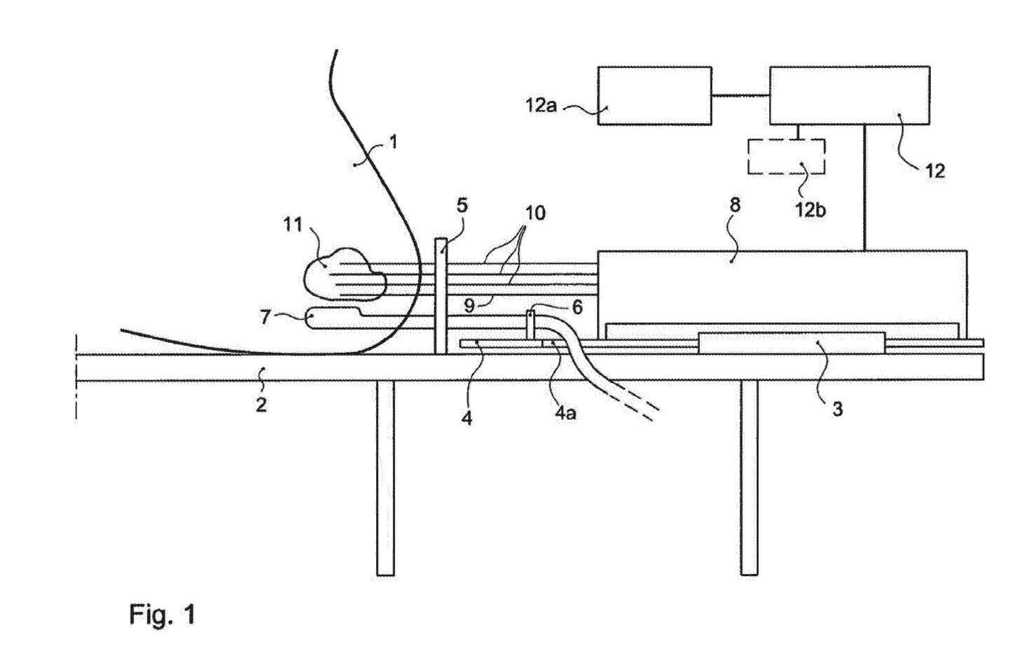

FIG. 1 shows an exemplary diagram of a known ultrasound system for implanting hollow catheters into a prostate gland. A patient 1 is shown lying in lithotomic position on a table 2. Fixedly connected to the table 2 is a housing 3. In one implementation, housing 3 may comprise a drive means 4 to move a rod 4a in a stepwise manner. A template 5 may be connected or mounted to table 2 and may include a plurality of guiding holes (not shown) through which hollow catheters 9, 10 can be positioned relative to the patient. By means of a holder 6, a trans-rectal imaging probe 7 may be fixedly connected to rod 4a, which may be moveable in a direction to and from the patient by means of drive means 4. In one embodiment, imaging probe 7 can be an ultrasound probe.

Catheter 9 may be used for fixating the target anatomical portion, for example, prostate gland 11, in position relative to template 5. A number of catheters 10 may be fixed into position through template 5 in prostate gland 11. Template 5 may thus determine the relative positions of catheters 10 in two dimensions. In HDR brachytherapy, all catheters may be closed at the distal end.

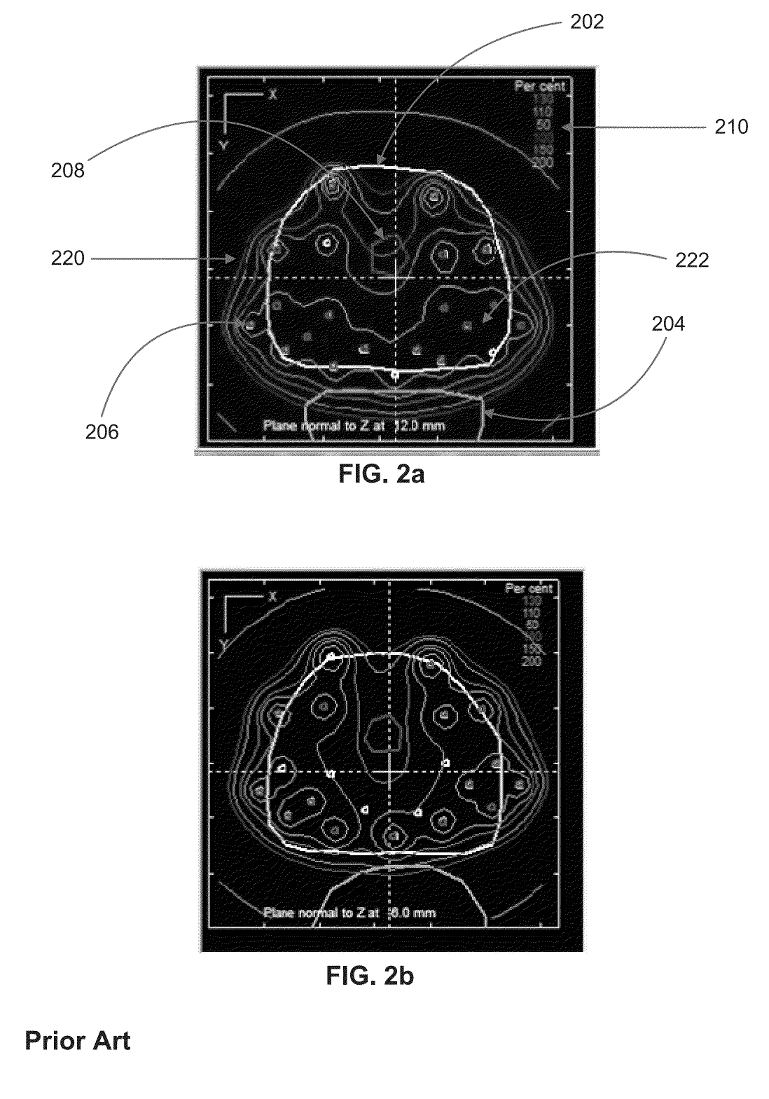

Because ultrasound images generated by the treatment delivery setup of FIG. 1 may be less suitable for treatment planning, the disclosed embodiments may use a CT or MR scanner (not pictured) on the patient, prior to treatment, in order to get detailed images of the target anatomical portion and surrounding organs, together with the implanted catheters. In one embodiment, patient 1 may be moved to the CT or MR scanner without being connected to the afterloader 8. Using the CT or MR images, the isodose lines of the target anatomical portion and the surrounding organs may be drawn in a set of parallel planes, transverse through the patient, typically 3 to 5 mm apart (see, e.g., line 220 in FIG. 2a). The isodose lines may represent lines connecting points of equal absorbed radiation doses in a radiation field (see dose percentages 210 in FIG. 2a). Drawing the contours around the target anatomical portion (e.g., line 202 in FIG. 2a) and organs at risk (e.g., line 204 in FIG. 2a, representing the rectum) may be done on the treatment planning module 12a or on a stand-alone treatment planning system (not shown) and then importing the dwell times to the control device 12 or to a stand-alone afterloader by a DICOM interface. Treatment planning program 12b, part of treatment planning module 12a, or the stand-alone treatment planning system may be used to optimize the dose distribution using the disclosed embodiments of EGO and IIP. Treatment planning program may contain instructions embedded on a memory device for execution by a processor of treatment planning module 12a or stand-alone treatment planning system. After optimization, patient 1 may then be treated using control device 12 by connecting patient 1 to afterloader 8, or by a stand-alone afterloader. The embodiment if FIG. 1 is not intended to be limited to the particular treatment set-up depicted. For example, the embodiments disclosed herein contemplate treating patients in a shielded room with an HDR or PDR afterloader.

The size and shape of the prostate determines the desired number and orientation of hollow catheters 10 as well as the relative positions of the energy emitting source(s) in each catheter 10 for displacement through catheter 10 towards prostate gland 11.

The following describes an example mode of operation of the device shown in FIG. 1. Patient 1 under anaesthesia lies on operating table 2. The (ultrasound) imaging probe 7 may be introduced into the rectum and connected via a signal line with an image screen, where an image may be seen of the inside of patient 1, in particular of prostate gland 11 as seen from the point of view of imaging probe 7. Template 5 may be attached to the perineum of patient 1 to prevent or minimize any relative movement of template 5 and prostate gland 11 and catheters 9, 10.

Drive means 4 may be used to move ultrasound probe 7 longitudinally and also to rotate it to provide different angular images. Prostate gland 11 may be fixed relative to template 5 by means of one or more catheters 9, 10. Subsequently further catheters 10 may be introduced into patient 1 and prostate gland 11 one by one under ultrasound guidance.

Moving imaging probe 7 with drive means 4 longitudinally and rotationally within the rectum will provide the necessary images. After all catheters 10 have been placed, their positions relative to prostate gland 11 may be determined in at least one of several known ways. The information from the planning module 12a about the displacement of the energy emitting sources through catheters 10 in terms of dwell positions and dwell times may then be used to control radiation delivery unit 8.

In the example embodiments, the energy emitting HDR/PDR source may be moved through catheters 10 in a discrete manner. For example, a stepping motor may advance the energy emitting source in a stepwise manner between subsequent dwell positions. The energy emitting source may be maintained in each dwell position for a certain dwell time. The dwell time for each dwell position in general determines the amount of radiation delivered at each dwell position. The radiation dose at individual or particular dwell positions may be considered as having a point, source-like distribution, where the peak of each radiation dose depends on the dwell time at the dwell position. The longer the dwell time, the higher the peak of the radiation dose at the dwell position.

To further refine the radiation dose delivered to patient 1, a user or clinician may use therapy planning module 12a or a stand-alone treatment planning system to optimize the dose distribution. In optimizing a dose distribution, two types of implants are distinguished: distance implants and volume implants. In a distance implant, the specified dose should be delivered at a determined distance around the catheters. Dose points may then be located at this given distance and the optimization aims to determine the dwell positions and relative dwell times such that the prescription isodose line 220 (the red line indicating 100% dosage in FIG. 2a) passes through these dose points. This may be referred to as “optimization on distance.” Examples of distance implants may be single catheters, double catheters, and single plane implants (e.g., moulds). In distance implants, dose points may be placed only relative to the catheters.

A volume implant contains one or more planes of catheters. In example embodiments, dose points may be placed inside the target anatomical portion and on the target contour lines. In a volume implant, the relative dwell times may be optimized to match different constraints to the different types of dose points. This may be called “optimization on volume.” In the example embodiments, the prescription dose is represented by prescription isodose line 220, and encompasses the target anatomical portion as closely as possible.

In cases where placement of dose points is complicated due to irregularities in the distances between implanted catheters 10, the dwell positions themselves can act as dose points for optimization. This technique may be called “geometric optimization” and can be performed either on distance or on volume.

Stage 1. Enhanced Geometric Optimization

In optimizing a dose distribution, two types of optimization methods may be distinguished: forward optimization and inverse optimization. When forward optimization is used, the user may optimize the dose distribution and then judge its suitability for treatment of the patient. Examples of forward optimization are geometric optimization and polynomial optimization using dose points, which are known in the art. Depending on the type of implant used, the aim of each of these optimization methods may differ. For a distance implant, the aim may be that the prescription isodose line should pass as closely as possible through each of the defined dose points (i.e., polynomial optimization). Alternatively, the aim may be that the prescription isodose line is located at the prescribed distance from the catheters (i.e., geometric optimization on distance). For a volume implant, the aim may be that the dose in the target anatomical portion midway between the catheters should be as homogeneous as possible throughout the implant (i.e., geometric optimization on volume). When X-ray films are used to reconstruct the positions of the catheters in the patient, the implant itself defines the target anatomical portion. In such a case geometric optimization may be the only optimization performed.

Geometric optimization (a type of forward optimization) uses each dwell position of an HDR or PDR source in an implant as a virtual dose point for the other dwell positions. GO then aims to achieve the same dosage value in each one of these virtual dose points. GO on volume increases the homogeneity of the dose distribution over the implanted volume, but the result may be of only of clinical value if the dwell positions in the catheters cover the target volume. In other words, if the implanted volume coincides with the target volume. Thus, GO and EGO optimizes the dose homogeneity of the dose distribution of the implanted volume and only when the implanted volume coincides with the target volume, will GO and EGO optimize the target volume. Clinical experience may be needed to judge the geometrically optimized dose distribution for each patient treatment. Based on that judgment, further changes can be made to the dose distribution, e.g. by manipulating dwell weights, resulting in a new dose distribution. Several iterations may be required before the final dose distribution will be accepted for clinical use.

The present disclosure pertains to an optimization tool for enhanced methods for geometric optimization (i.e. “enhanced geometric optimization” or “EGO”), using two parameters to optimize the dose distribution: (1) the volume strength and (2) the volume range. The EGO tool allows a clinician or other treatment provider to adjust these two parameters to vary the dose distribution to an implanted volume in order to achieve an optimal dose distribution. By using the EGO tool, the user can create the optimal dose distribution using a treatment planning system or therapy planning module 12a, and the optimal dose may then be delivered to patient 1 via a stand-alone afterloader or treatment unit 8.

When a user increases the volume strength parameter of a dose distribution, the adjustment will enlarge the transport of dwell weight from the center of the target anatomical portion to the outer regions of the implanted volume. Additionally, the user may adjust the volume range parameter in implants where converging catheters come together at one end or when the distances between the catheters is about the same magnitude as the dwell step, when catheters cross one another. When this occurs and the volume range parameter is not adjusted, the target anatomical portion around the catheters may become increasingly under-dosed as the distances between the catheters decreases.

FIG. 2a illustrates an exemplary screen shot of an HDR prostate implant with the dose distribution in the CT-slice Z=12 mm with all dwell weights set to 1.0, as is known in the prior art. The 100% prescription dose (PD) isodose line 220, encompasses the target anatomical portion (or “clinical target volume” (CTV)), but this dose distribution also delivers a high dose to rectum 204, up to 150% of the PD, which is represented by the 150% line from table 210 lying within rectum 204. FIG. 2a also demonstrates a large contiguous volume 222 having a value for the dose limit of 200% (see table 210), which may be too high for an optimal dose. FIG. 2b illustrates an exemplary screen shot of the PDR prostate implant, using geometric optimization (as is known in the prior art), with an increased dwell time taken from the center of the implant and moved to its periphery. Despite the optimization procedure employed in FIG. 2b, the dose distribution may still not be optimal because, while the dose to the rectum is reduced, it remains more than 100% of the PD, which is represented by line 220 lying within rectum 204 in FIG. 2b. Also the dose to urethra 208 is reduced from 130% in FIG. 2a to only just below 130% in FIG. 2b, which is represented by urethra 208 lying with the 110% isodose line (see table 210).

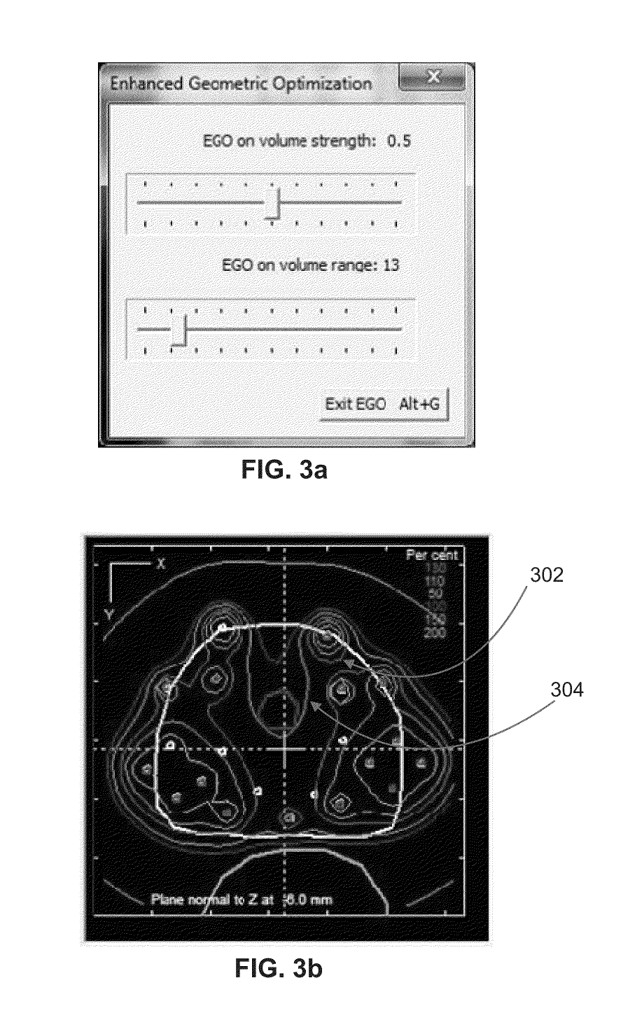

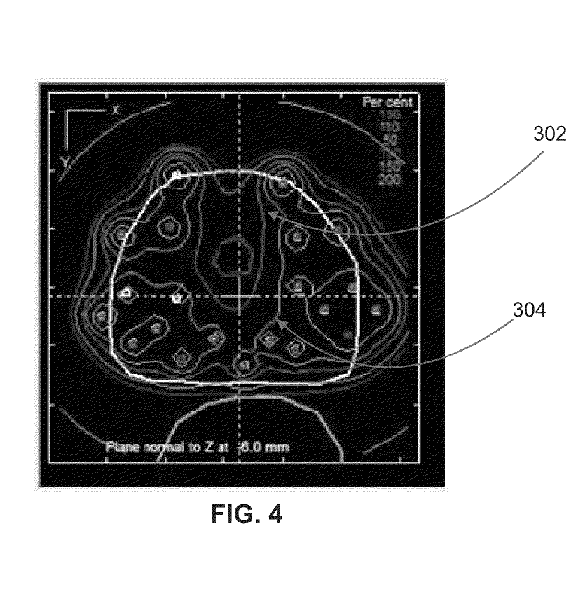

As a result, the user may want to employ an EGO tool, consistent with the example embodiments, for optimizing the dose distribution by further enhancing the GO dose distribution. FIG. 3a illustrates an exemplary user interface tool, e.g., a slider scale of volume strength and volume range, that may be used when employing EGO, according to the disclosed embodiments. In one embodiment, the slider scale may be provided in a user interface tool on the treatment planning system, e.g. therapy planning module 12a, providing a digital interface that may be adjusted by a user (via clicking, swiping, touching, etc.). The volume strength parameter may be in the range of 0.0-1.0 and steers the amount with which a dwell weight, as obtained by GO, is increased or decreased. The volume strength parameter with the value “s” sets each dwell weight to the value: dwell weight by GO−(1.0−dwell weight by GO)*s). The EGO algorithm loops over all dwell positions and each dwell position acts as a dose point for all other dwell positions.

In the example shown, the volume range parameter is the radius in millimeters of a sphere around each dwell position when acting as a dose point. A volume range parameter may be used if the implant is poorly placed but cannot be moved. In that situation, a catheter may be so near to another catheter that there is no longer a dwell weight. The volume range parameter thus reflects a distance or range around a given dwell position acting as dose point for the other ones, in which the dose from any other dwell position within that distance or range is not allowed to contribute to the dose in this given dwell position when employing the EGO algorithm. When the volume range parameter is adjusted, the dose contributions to the dwell position that is acting as a dose point at the center of the sphere (created via the adjusted distance or range) may be summed, disregarding the dose contribution from any dwell position that is situated inside this sphere. In this example, the volume strength parameter is adjusted to 0.5 and the volume range is adjusted to 13. As a result, the dose to the rectum is reduced even further than when normal geometric optimization (FIGS. 2a-b) is used. When the volume strength is increased using the EGO tool, dosage moves from the inside of the implant to the outside of the implant, further optimizing the dose distribution, until the maximum homogeneity of the dose distribution of the implanted volume may be obtained. For example, the user may want to maximize the value of the dose-volume-histogram parameter Quality Index (QI), which is a measure of the dose homogeneity over the implanted volume. FIG. 3b illustrates an exemplary screen shot of the HDR prostate implant when employing the EGO values of FIG. 3a. FIG. 3b illustrates a cross-section of the HDR prostate implant that has been adjusted using the exemplary slider scale of FIG. 3a for EGO. The cross-section of the implant may be generated using the imaging probe 7 or a CT or MRI scan. By making this adjustment, the dose to the rectum 204 is now less than 100% of the PD, and the dose to the urethra 208 is at about the optimal value of 110% PD. Also the dose is increased in the upper part of the target anatomical portion, as illustrated by lines 302 and 304 (representing 130% and 110% PD, respectively), making the dose distribution to the target more homogeneous.

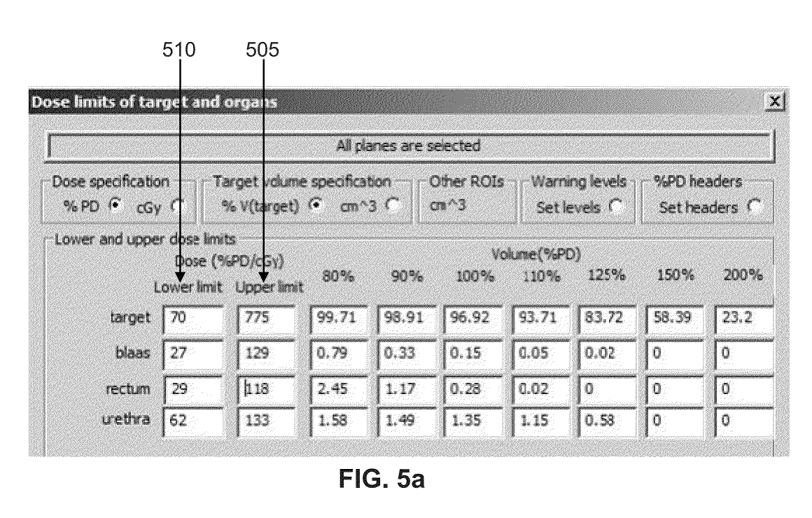

FIG. 4 illustrates an exemplary screen shot of the HDR prostate implant, employing EGO, according to the disclosed embodiments. In FIG. 4, the volume strength has been adjusted from 0.5 (FIG. 3b) to 0.3, using the exemplary slider scale of FIG. 3a. As a result of adjusting the volume strength to a lesser amount (0.3), the cross-section of the HDR prostate implant illustrates that the dosage moves from the outside of the implant toward the inside of the implant, illustrated by lines 302 and 304 again, which in this case may be less optimal. As a result, the dose to the rectum and to the urethra is increased.

A user or clinician can vary the volume strength and range values to determine the optimal dose distribution to an implanted volume. The user may adjust the values using a user interface device on the treatment planning system, e.g. therapy planning module 12a. The adjustments may cause the system, such as therapy planning module 12a, to generate isodose lines depicting the dose distribution to a CTV based on the received parameters. The user may then examine the resulting isodose lines on, for example, a display of therapy planning module 12a. The display may include a cross-section of the implant, depicting the dwell positions and target isodose lines surrounding the CTV, such as in FIGS. 3b and 4.

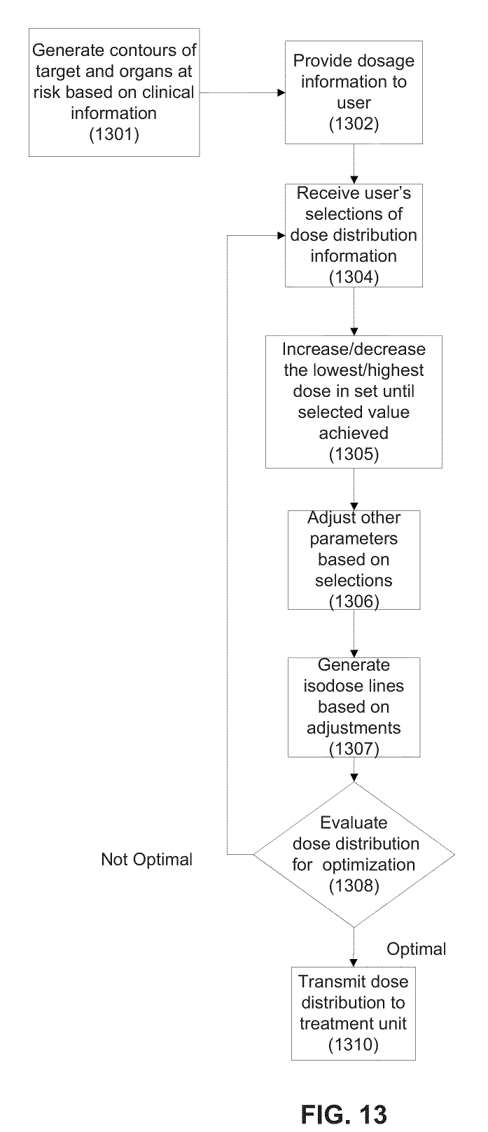

FIG. 12 illustrates an exemplary method for optimizing a dose distribution around implanted catheters through the iterative optimization algorithm of enhanced geometric optimization, according to the disclosed embodiment. In step 1201, the isodose distribution may be generated based on clinical information. The information may include the relative positions of the sources in each catheter for stepping through the catheter within, for example, prostate gland 11. In step 1202, information about the dose distribution to an implanted volume (treatment planning based on radiographs) or a target anatomical portion (treatment planning based on CT or MR slices) may be provided to the user. The information may be visualized by the treatment planning system, e.g. therapy planning module 12a. The information may also include isodose lines based on the current dose distribution. The information may be made available to the user via a user interface tool or a display device associated with the treatment planning system, e.g. therapy planning module 12a.

In step 1204, the system may receive a selection of the dose distribution information, such as the volume strength or the volume range. The system receiving the selection may include the treatment planning system, e.g. therapy planning module 12a. For example, the user may enter his selections of volume strength or volume range (or both) via a user interface tool associated with the treatment planning system, e.g. therapy planning module 12a.

In step 1205, the system may adjust the dose distribution to the implanted volume, based on the received selections. For example, the system may receive a value for the volume strength of the dose distribution and may enlarge the transport of dwell weight from the center of the implanted volume to the outer regions of the implanted volume, using the EGO algorithm. The EGO algorithm may loop over all dwell positions using each dwell position as a dose point for all other dwell positions, and may increase or decrease the dwell weight, using: dwell weight by GO−(1.0-dwell weight by GO)*s), where the value “s” is the received selected value.

In step 1206, the system may generate isodose lines for the dose distribution of the target anatomical portion and any organs proximate to the target anatomical portion (e.g., organs at risk of being affected adversely by the dose distribution), based on the received selections of the distribution information.

In step 1208, the dose distribution may be evaluated by analyzing the generated isodose lines on, for example, a set of anatomical cross-sections with the isodose lines of both the CTV and the organs at risk. These anatomical cross-sections may be generated before the optimization of the dose distribution started, and the treatment planning system may overlay the isodose lines of the target anatomical portion and organs at risk. For example, the user may view a set of cross-sections of the implant (such as the one in FIG. 3b), which shows the isodose lines and the dwell positions in the plane of planning. In one embodiment, it may be determined that the dose distribution is not optimal; in other words, that the homogeneity of the dose distribution over the implanted volume is not optimal. Thus, the distribution information again may be adjusted, which may be received by the system, as described in step 1204. As the dose distribution over the implanted volume may now be considered optimal, the next step may be to determine whether the dose distribution covers the target volume and/or whether the dose to an organ at risk is too high. For this step the Interactive Inverse Planning can be used. However, if the dose distribution is optimal (step 1210), the system may transmit the dwell weights and the normalization of dwell times to the radiation unit 8 for use in treating the target anatomical portion 11, according to the values received in step 1204.

Stage 2. Interactive Inverse Planning

In the example embodiments, the next stage of the present disclosure for optimizing a brachytherapy dose distribution may be interactive inverse optimization IIP. However, this real-time optimization method may also be suitable for finalizing a dose distribution obtained with an optimization method other than EGO. With an inverse optimization (“IO”) tool, the user or clinician may specify the dose limits of the target anatomical portion and the organs at risk surrounding it. The IO tool may then attempt to optimize the dose distribution to the target anatomical portion and the organs at risk based on the specified dose limits. Exemplary inverse planning procedures used in previous techniques include inverse planning by simulated annealing (“IPSA”) and an hybrid inverse planning optimization algorithm (“HIPO”).

Previous inverse planning programs, such as IPSA or HIPO, are based on specifying the penalties for exceeding required properties of the brachytherapy dose distribution in treating the target anatomical portion. These prior procedures required that a user or clinician specifies all properties simultaneously, which results in a single solution (IPSA) or a set of clinically acceptable solutions (HIPO).

An IIP tool consistent with example embodiments also allows optimization of a dose distribution. By using the IIP tool, the user may specify a single property of the dose distribution, and the resulting dose distribution may be displayed in real-time. For example, the user may specify the dose distribution using a user interface tool available on, for example, therapy planning module 12a. The resulting display may include a cross-section of the implanted volume displaying the isodose lines of the CTV and the surrounding organs. After inspecting the resulting dose distribution on the display, the user can change the same or another property and again, in real-time, inspect the result on the display. As an example, in an HDR prostate implant, the clinician may want to reduce the dose to the rectum to 80% of the prescription dose (PD). By reducing the rectum dose, the dose to the target anatomical portion nearest to the rectum subsequently reduces to 85% PD. The clinician can now judge whether this dose distribution is optimal for this particular patient (e.g., by understanding where the tumor is located in the patient's prostate gland and where the radiation needs to be delivered to the specific patient) or whether the maximum dose to the rectum or the minimum dose to the target anatomical portion nearest to the rectum must be changed again.

When using the IIP tool, the user may first optimize the dose distribution by using, for example, the EGO tool (FIGS. 3a-b), to create a dose as homogeneous as possible, or by IPSA or HIPO, as described above. The user may then use the IIP tool to fine-tune the already optimized dose distribution. For example, while the dose to the target anatomical portion may be optimized, the user may determine that this optimal dose distribution too greatly affects the surrounding organs. Thus, the user may be willing to sacrifice the homogeneity of the dose distribution (achieved through EGO) throughout the target anatomical portion to some extent in the areas nearest the organs at risk in order to reduce the dose to these at-risk organs, further optimizing the dose distribution. In other embodiments, the user may not use any forward optimization procedures (like EGO) or inverse optimization procedures (like IPSA or HIPO); in other words, the user may start with all dwell weights at 1.0 before employing IIP.

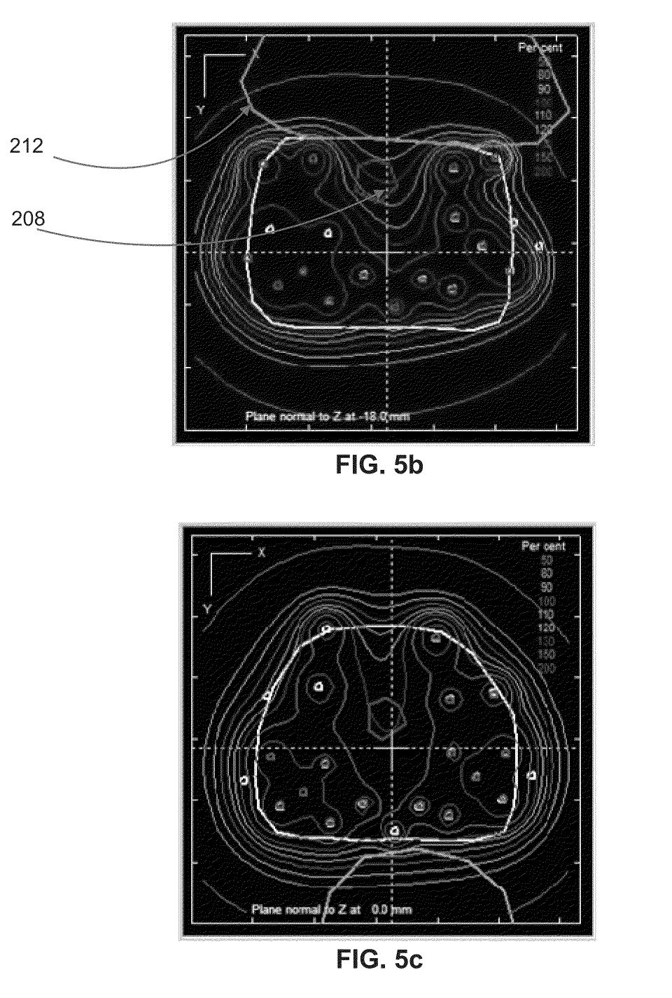

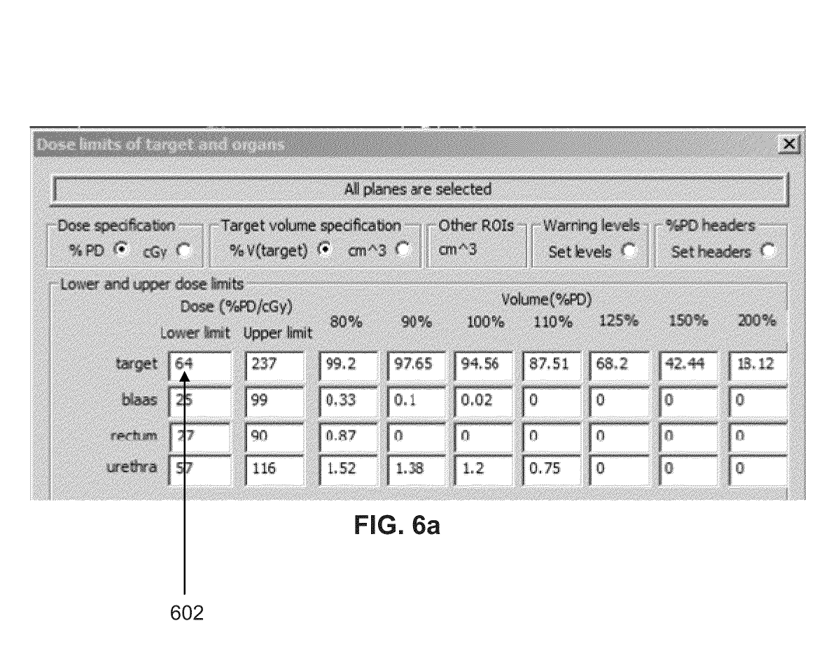

FIG. 5a illustrates an exemplary table of dose limits of a target anatomical portion and its surrounding organs, according to the disclosed embodiments. In this example, the first step of the EGO tool (FIGS. 3a-b) has already been employed by the user to adjust the volume strength to 0.3 and the volume range to 12 using, for example, the exemplary user interface tool illustrated in FIG. 3a. However, in this example, the dose to the organs at risk, the bladder 212 (FIG. 5b) and urethra 208 (FIG. 5b), may be considered to be too high, which is evidenced by the upper limits of 129% PD and 133% PD, respectively, in FIG. 5a. FIGS. 5b and 5c illustrate isodose lines for this dose distribution generated according to the treatment planning parameters disclosed by FIG. 5a. FIG. 5b illustrates the cross-section when the plane is normal to Z at −18.0 mm, i.e. at the area where bladder 212 is located above prostate 202, and FIG. 5c illustrates the plane normal to Z at 0.0 mm, where rectum 204 is located under the prostate. Thus, FIGS. 5b-c are different cross-sections of the same implant. The user may determine that the doses to the organs at risk seem too high (e.g., not optimal) by viewing the isodose lines corresponding to the upper limit values 505 indicated in FIG. 5a. In this figure the upper limit to the rectum (118% of the prescription dose) may be too high. As shown in FIG. 5c, the 110% and 100% isodose lines indeed intersect rectum 204 in this image, and a significant volume of the rectum will receive a dose much higher than the clinically acceptable value of 90% PD.

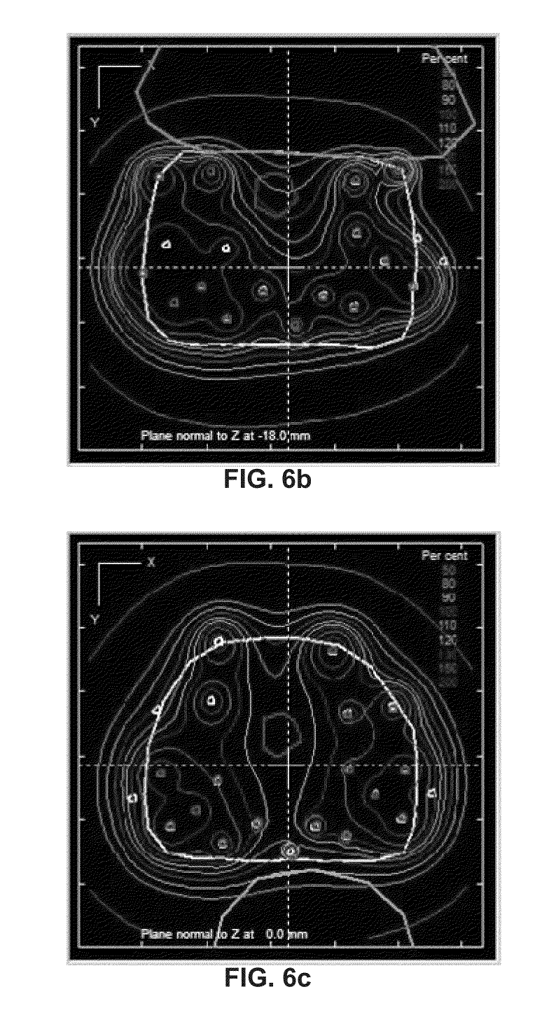

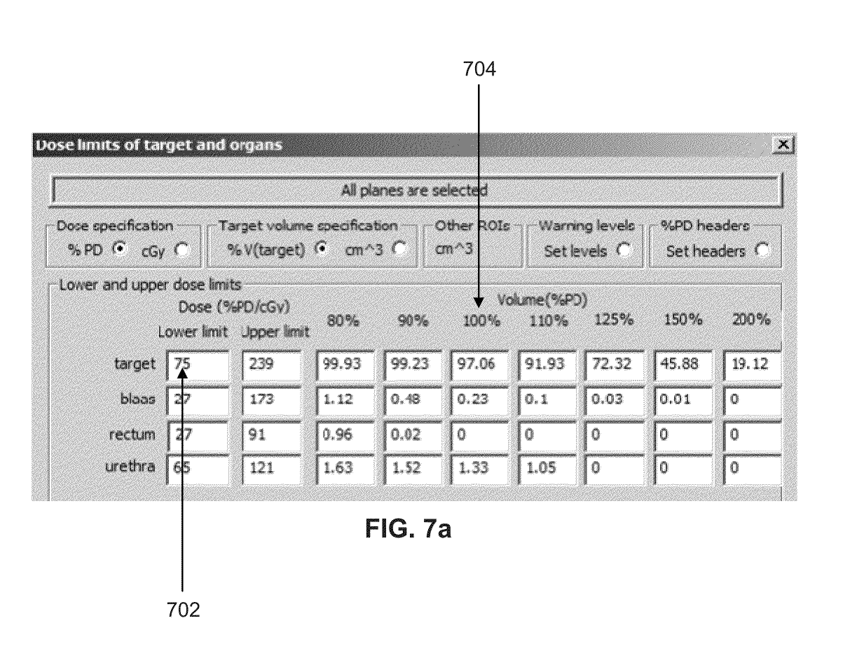

Thus, the user may use the IIP tool to further optimize the dose distribution, by adjusting one or more upper or lower limits of the dose volumes in the clinical target anatomical portion and the organs at risk. FIG. 6a illustrates an exemplary table of dose limits of the target anatomical portion, optimized to a clinically defined level, according to the disclosed embodiment. The display may include a digital user interface tool for the user to input selections (i.e. by clicking, swiping, touching, etc.). If the user changes one of upper limits 505 or lower limits 510, that selection will be obtained, but the other limits of the target anatomical portion or the organs at risk will also be influenced. Therefore, FIG. 6a displays the values that the system actually obtains for the upper and lower limits of the target anatomical portion and the organs at risk, based on the following user's selection of the upper limits successively: urethra at 120%; bladder at 100%; and rectum at 90%. After the user has made these three selections, the system obtains the upper and lower limits of the prescription dose given in FIG. 6a (e.g., upper limit of 116% to urethra, 99% to bladder, and 90% to rectum). As a result, the system generates the isodose lines based on the dose settings in FIG. 6a, which are displayed in FIG. 6b. By viewing FIG. 6b, it becomes clear that the bladder 2121 lies against the prostate 202 when the dose to the bladder was set to 100% using the tool of FIG. 6a. Therefore, the lower dose limit of the target anatomical portion will have a low value, here 64% of the prescription dose (see 602 in FIG. 6a). Next, the treatment planner may increase the lower dose limit to the target anatomical portion using the tool in FIG. 6a and, in real-time, evaluate how the upper limit value of the dose to the bladder changes, by viewing FIG. 6a or an image as in FIG. 6b-c. In the case of HDR or PDR prostate implants, the limits essential for optimization include the lower limit to the target anatomical portion and the upper limits to the organs at risk, and may also include the upper limit to the target anatomical portion. The treatment planner interactively changes the value of an upper or lower limit to either the target anatomical portion or the organs at risk and, in real-time, evaluates the resulting dose distribution via both the generated isodose lines together with the updated display of dose limits of target anatomical portion and organs at risk in the tool of FIG. 6a. In the case of FIGS. 6a and 6b, where reducing the upper limit to the bladder conflicts with the lower limit to the target anatomical portion, the IIP optimization algorithm detects where the bladder lies against the target anatomical portion and changes only the dose in the volume adjacent to the bladder. In this way the reduction of the dose to the target anatomical portion may be minimized while still reaching the lower limit set to the bladder. The isodose lines of the target anatomical portion and organs at risk may be viewed by the user on a display on therapy planning module 12a. These isodose lines are usually superimposed on a set of cross-sections of the target anatomical portion generated by, for example, imaging probe 7. FIGS. 6b and 6c illustrate the isodose lines generated according to the treatment planning settings of the technique disclosed by FIG. 6a. FIG. 6b illustrates the cross-section when the plane is normal to Z at −18.0 mm, and FIG. 6c illustrates the plane normal to Z at 0.0 mm. Thus, FIGS. 6b-c are different cross-sections of the same implant. In this example, the user may view the isodose lines illustrating the dose distribution over the target anatomical portion, rectum, urethra, and bladder based on the user's selections of lower limits of 90% for the rectum, 100% for the bladder, and 120% for the urethra. As a result of these settings, the lower dose limit for the target anatomical portion is adjusted to 64%.

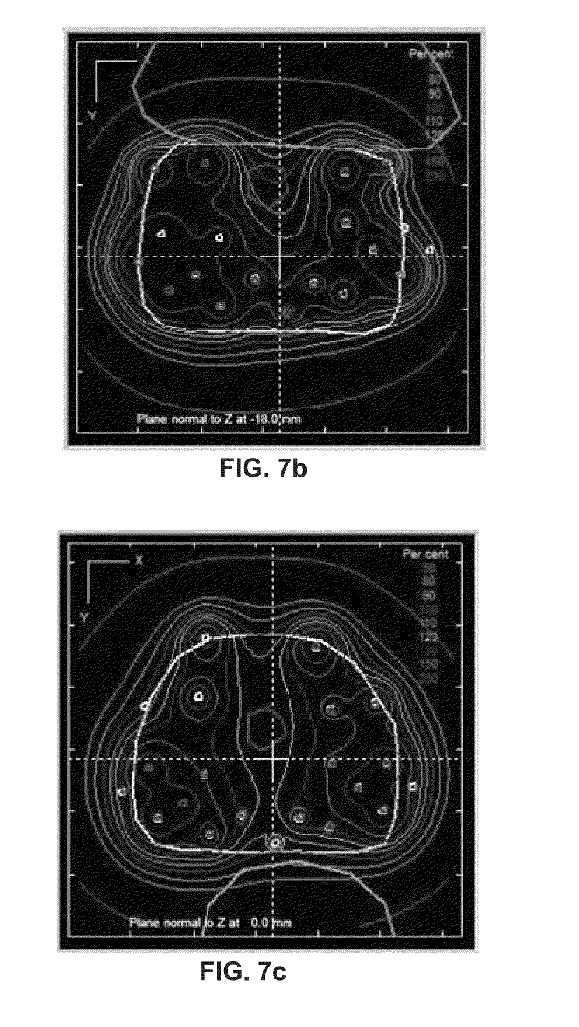

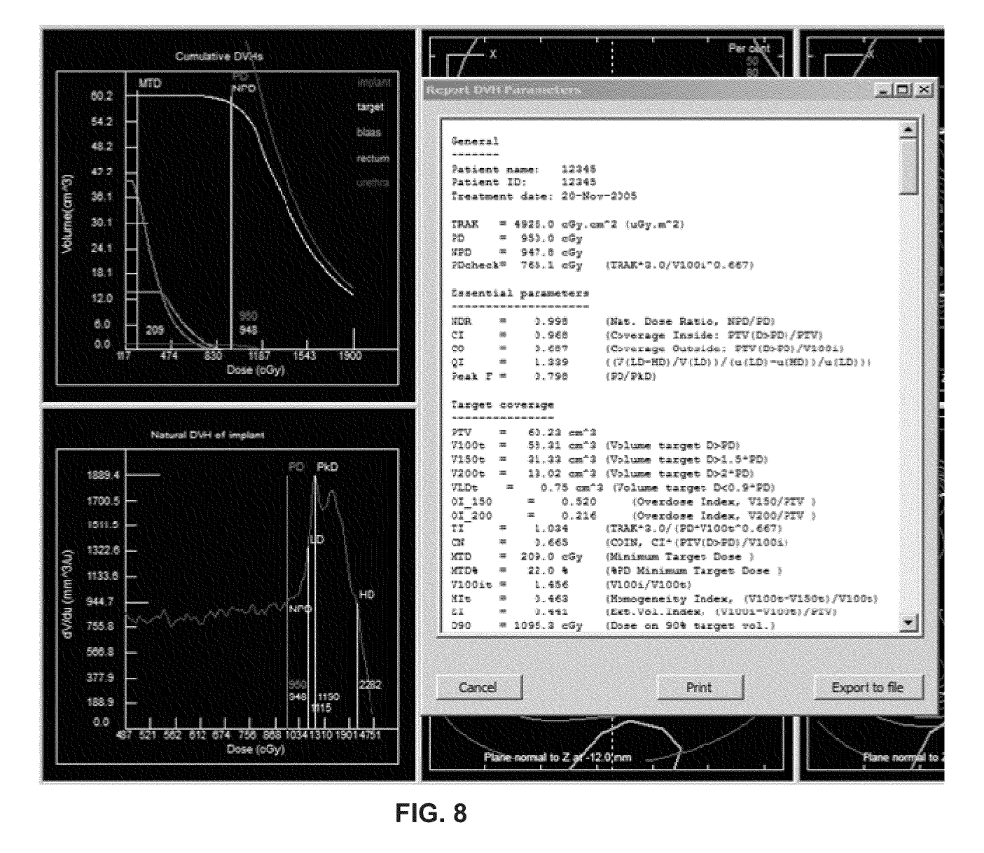

If the user considers the lower limit to the target anatomical portions too low, after inspecting the dose distributions, the user may adjust the dose distribution to the target anatomical portion to, for example, 75% coverage by setting this value in field 702 (FIG. 7a) for the lower limit to the target. FIG. 7a illustrates an exemplary table of dose limits of the target anatomical portion, according to this disclosed embodiment. In this example, the user may set the lower limit 702 for the target anatomical portion to 75%. By setting this lower limit, the treatment planning system, e.g. therapy planning module 12a, may increase the highest dose to the bladder to 173% of the PD. As a result, the volume of the target anatomical portion that has at least 100% of the PD (704) increases from 94.56 (FIG. 6a) to 97.06 cm³ (FIG. 7a). Therapy planning module 12a may generate isodose lines based on the user's selections of the lower limit for the target anatomical portion and the system's adjustment to the other limits based on the lower limit selection. FIGS. 7b and 7c illustrate the isodose lines generated according to the treatment planning settings of the technique disclosed by FIG. 7a. FIG. 7b illustrates the cross-section when the plane is normal to Z at −18.0 mm, and FIG. 7c illustrates the plane normal to Z at 0.0 mm. Thus, FIGS. 7b-c are different cross-sections of the same implant. In clinical practice, the user views on a monitor a set of cross-sections, typically 2-5 mm apart, through the whole target anatomical portion. In this example, the user may view the cross-section of the target anatomical portion and the isodose lines generated according to the treatment planning settings of the technique disclosed by FIG. 7a.

Thus, by adjusting the values of the lower and upper limits, using the user interface tool on therapy planning module 12a, the user can determine the resulting dose distribution practically instantaneously by viewing the generated dose distribution illustrated by generated isodose lines on an image. Additionally, the user can make further adjustments to the various parameters to optimize the dose distribution.

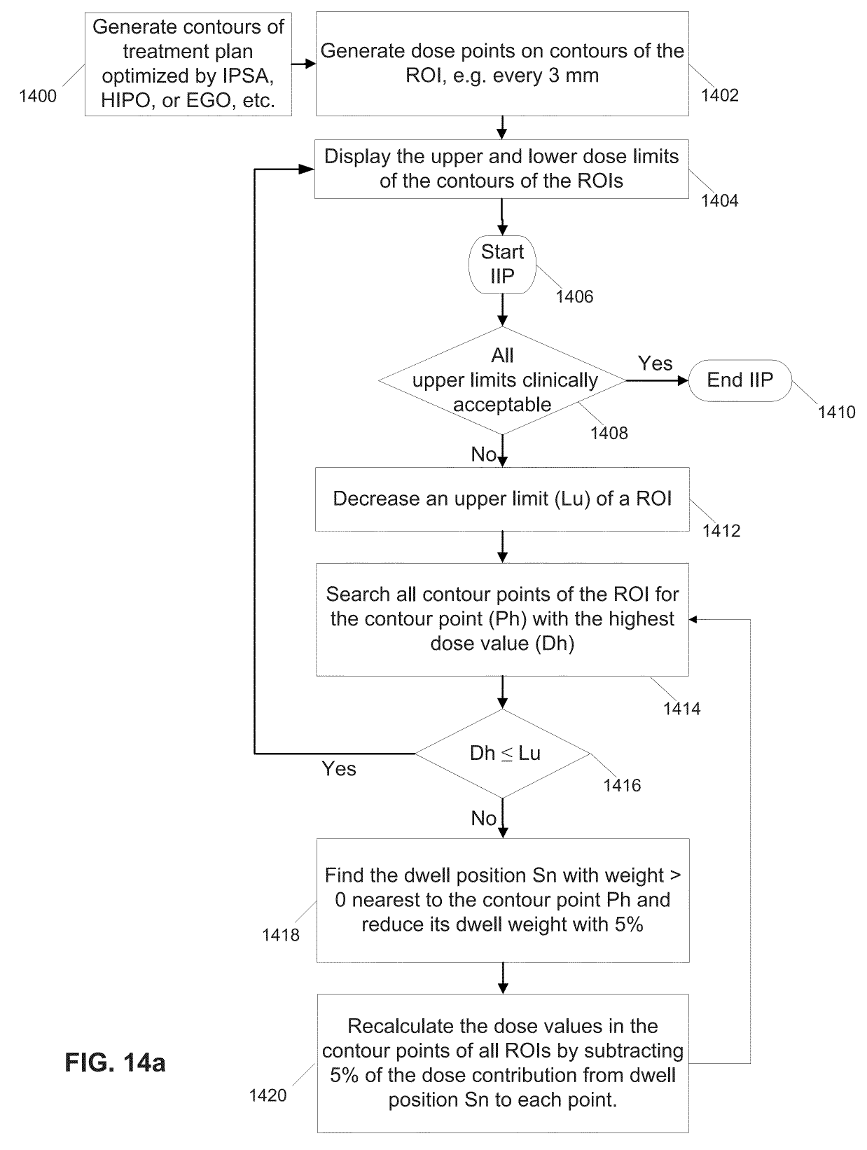

FIG. 13 illustrates an exemplary method for optimizing a dose distribution through interactive inverse planning (IIP), according to the disclosed embodiment. In step 1301, contours of targets and organs at risk may be generated based on clinical information. The information may include isodose lines, based on the clinical information, and the relative positions of the sources in each catheter for stepping through the catheter within, for example, prostate gland 11. In step 1302, information about the dosage to a target anatomical portion and the organs at risk may be provided to the user. The information may be provided by the therapy planning module 12a or the therapy planning program 12b. The information may also include the dose volumes and ranges of the target anatomical portion and organs at risk. The information may be made available to the user via a user interface tool or a display device associated with the treatment planning system. The information may include a table of dose limits for the target anatomical portion and the organs at risk, such as that depicted in FIG. 7a, for adjusting the upper and lower limits of a dose. The information may also include a set of randomly placed dose points in the regions of interest (ROI), e.g., the target anatomical portion and the organs at risk.

In step 1304, the system may receive the selections of the dose distribution information, such as the upper limit or the lower limit of the target anatomical portion or an organ at risk. The system receiving the selections may include the treatment planning system. For example, the user may enter his selections via a user interface tool, such as a digital display device (i.e., clicking, swiping, touching, etc.).

In step 1305, the interactive inverse optimization algorithm of IIP may be executed. For each limit set for the target (i.e., 702) or organ at risk in FIG. 7a, the next loop within square brackets may be iterated until the limit is met.
(1) Upper limit. [For the upper limit set for a given region of interest (ROI), the highest dose value may be searched along all contour points of that ROI. If the highest dose value is lower than or equal to the limit value, exit the loop. If the highest dose value exceeds the limit value, find the nearest dwell position with weight greater than zero, and reduce that weight with, for example, 0.05.].
(2) Lower limit. [For the lower limit set for a given region of interest (ROI), the lowest dose value may be searched along all contour points of that ROI. If the lowest dose value is higher than or equal to the limit value, exit the loop. If the lowest dose value is lower than the limit value, find the nearest dwell position with weight greater than zero, and increase that weight with, for example, 0.05.]

Thus, the system may iteratively adjust the lowest (or highest) dose found on the contour of the ROI with the upper (or lower) limit investigated, until the selected value of the dose distribution has been achieved. For example, if the user sets the lower limit for the target contour to 75%, the treatment planning program may (in step 1305) increase (or decrease) the lowest (or highest) dose in the set of contour points until the dose to the target contour is above 75% throughout. Alternatively, if the user sets the upper limit for an organ at risk (e.g., the bladder) to 100%, the treatment planning system may (in step 1305) decrease the highest dose in the set of contour dose points until the dose to the bladder is less than 100% throughout. For example, the treatment planning system may search all contour points of the ROI for the point with the highest dose value, continuing to adjust the lowest or highest dose found on the contour by reducing or increasing the dwell time of the nearest dwell position with 5% until the limit has been reached.

In step 1306, the system may adjust other parameters to account for the received selections. For example, if the user sets the lower limit for the target anatomical portion to 75%, the treatment planning system may as a result (in step 1306) increase the highest dose to the bladder to 173% of the PD. As a result, the total volume of the target anatomical portion that has a dose of at least 100% PD may be adjusted to 97.06% of the target anatomical portion.

In step 1307, the system may generate isodose lines, based on, for example, the parameters of steps 1305 and 1306.

In step 1308, the dose distribution may be evaluated over the target anatomical portion and organs at risk. For example, the user may evaluate the dose distribution by viewing the generated isodose lines on, for example, a set of cross-sections of the implant in the target anatomical portion. For example, the user may view the dose distributions generated in FIGS. 7b-c. The isodose lines may be superimposed on a set of cross-sections of the target anatomical portion, generated by, for example, CT or MR scans. In one embodiment, the dose distribution may be determined to be non-optimal. For example, the user may determine that an organ at risk may be adversely affected by the current dose distribution to the organ. Thus, the dose distribution information may again be adjusted by altering the upper or lower limits, which may be received by the system, as described in step 1304. However, if the dose distribution is optimal (step 1310), the system may transmit the dose distribution to the treatment unit for use in treating the target anatomical portion 11, according to the values received in step 1304.

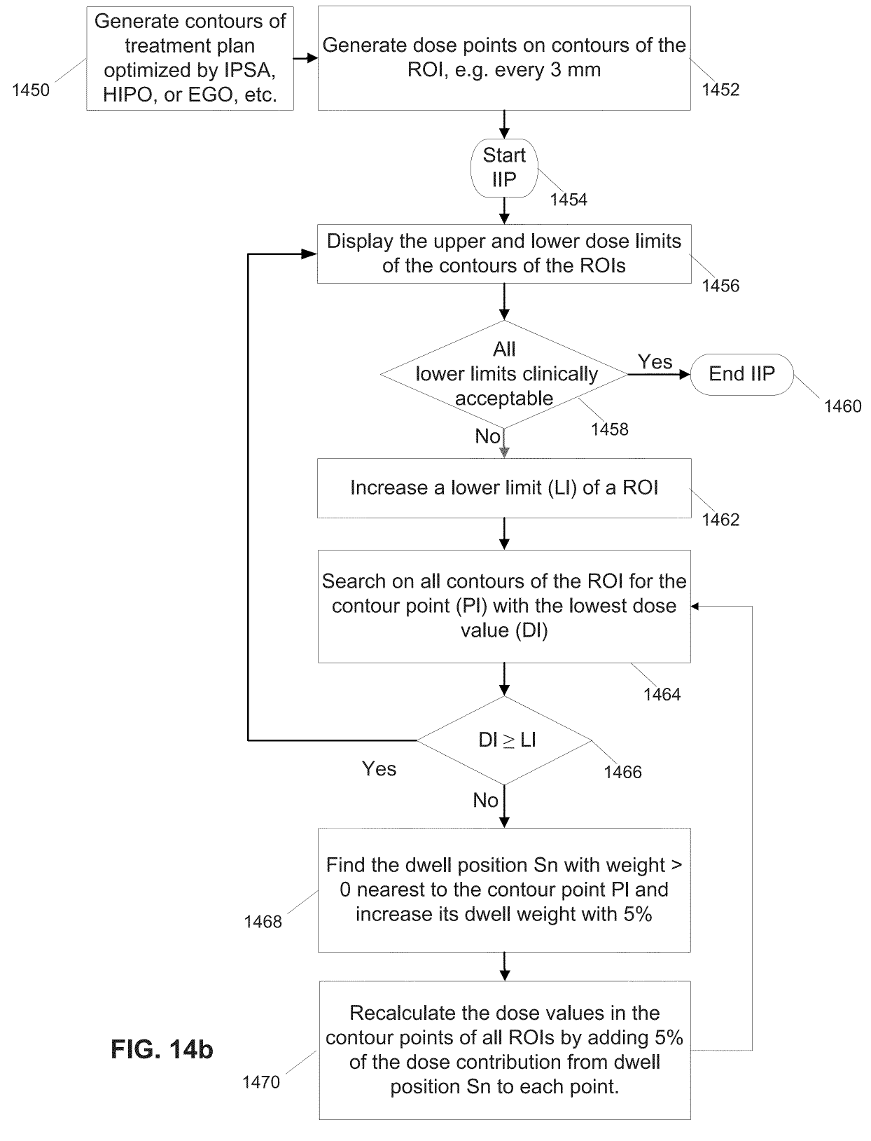

FIGS. 14a-b generally illustrate the process, as described above, for performing interactive inverse planning. As shown, and described above, steps 1400-1420 describe how the interactive inverse optimization algorithm of IIP may be executed for setting the upper limit for the target (i.e., 702) or organ at risk. Steps 1450-1470 describe how the interactive inverse optimization algorithm of IIP may be executed for setting the lower limit for the target (i.e., 702) or organ at risk. The values depicted in FIGS. 14a-b are merely exemplary and are not intended to limit the scope of the disclosed embodiment. For example, dose points may be placed apart from each other by 5 mm in step 1402 or 1452. In another embodiment, the dwell weight may be reduced or increased by 3% in 1418 or 1468, respectively.

Evaluating HDR and PDR Dose Distributions

In previous techniques, brachytherapy treatment plans are evaluated by an experienced brachytherapy treatment planner (e.g., a medical dosimetrist) using one or more treatment plan indices, graphs, and/or tables to determine the quality of the implant or application. The final evaluation may be done by the physician (e.g., a radiotherapist or radiation oncologist).

For example, brachytherapy treatment plans have been evaluated using one or more treatment plan indices based on Cumulative DVHs and the Natural DVH of the implanted volume. The Natural DVH may be used to evaluate the quality of the implant, i.e. the homogeneity of the dose distribution of the implanted sources. The Cumulative DVHs may be used to evaluate the matching of the dose distribution to the target anatomical portion and to the organs at risk.

Users may employ different sets of parameters to evaluate dose distributions. The following aspects of a dose distribution in brachytherapy may be taken into account: (1) coverage of the target anatomical portion, (2) conforming the dose to the target anatomical portion such that normal tissues near the target anatomical portion get a dose as low as possible, (3) keeping the dose in organs at risk below a given level, and (4) dose homogeneity throughout the target anatomical portion. Implant parameters derived from the NDVH and CDVH that are used in previous techniques include: 1) QI: Quality Index; 2) UI: Uniformity Index; 3) CI: Coverage Inside Index; 4) CO: Coverage Outside Index; 5) NPD: Natural Prescription Dose; 6) NDR: Natural Dose Ratio; 7) COIN: Conformation Index; and 8) DNR: Dose Non-uniformity Ratio. In the example embodiments, QI is derived from the NDVH and is a direct measure of the dose homogeneity over the implanted volume. Also in the example embodiments, UI combines the QI, which is related only to the implanted volume, with the Prescription Dose, which is related to the target anatomical portion. The combined value is usually between 1.0 and 1.5 for an optimized HDR prostate implant. Instead of using UI, it may be easier and more informative to use QI and the Natural Dose Ratio. CI may reflect the volume of the Planned Target Volume (PTV) with a dose greater than PD divided by the whole PTV, i.e., CI=PTV(D>PD)/PTV. CO may reflect the volume of the Planned Target Volume (PTV) with a dose greater than PD divided by the implanted volume encompassed by the PD isodose surface V100i: CO=PTV(D>PD)/V100i; NPD may be the prescription dose encompassing the implanted volume. In the Natural Dose-Volume Histogram (NDVH), the NPD may be located at the base of the peak representing the implanted volume with the dose distribution over this volume as homogeneous as possible. The more homogeneous the dose distribution, the larger and steeper the peak may be. NDR is the ratio of the NPD and the PD: NDR=NPD/PD. If NDR is greater than 1.0, and if the PD gets the clinical prescription dose value, the NPD will be larger than the clinical prescription dose and the implant as a whole will receive a larger dose than the clinical prescription dose. The risk of over-dosage increases. Whereas if NDR is less than 1.0, the risk of under-dosage increases. COIN is defined as COIN=CI*CO. In the example embodiments, an ideal situation may be CI=1, CO=1. DNR represents the ratio of the implanted volume with 1.5*PD/implanted volume with PD.

The present disclosure aims to evaluate brachytherapy treatment plans using a subset of the existing parameters derived from the Natural DVH and the Cumulative DVHs (discussed above), together with new parameters derived directly from the implant, such as the TRAK Index (“TI”), which is based on the Total Reference Air Kerma given to the implant normalized to the implant volume and the prescription dose.

The present disclosure may use the following five indices together for a user to evaluate (1) how well the implanted volume coincides with the target anatomical portion, (2) whether the target anatomical portion may be partially underdosed or overdosed, and (3) whether healthy surrounding tissues partially overdosed:
NDR: Natural Dose Ratio
CI: Coverage Inside Index
CO: Coverage Outside Index
QI: Quality Index
TI: TRAK Index

The Cumulative DVHs of organs at risk may determine the volumes in these organs receiving at least a given dose. It may make a clinical difference if the total volume of an organ receiving a given limit of a dose consists of a single contiguous volume or of a large set of small contiguous volumes, e.g., volumes around each dwell position. If the total volume of an organ receiving a given dose limit consists of a single contiguous volume (see 222 in FIG. 2a), the radiation dose to that volume may affect normal tissue in addition to cancerous tissue, and the normal tissue may not be able to repair the damage caused by the radiation. Therefore, it may be important to determine whether the dose distribution results in a single contiguous volume, as described above, or a set of smaller contiguous volumes, which may prevent or limit the damage to normal tissue.

In the present disclosure, the treatment planning system may generate graphs of the NDVH and the CDVH of the implanted volume, the target anatomical portion, and the volumes of the organs for implant evaluation.

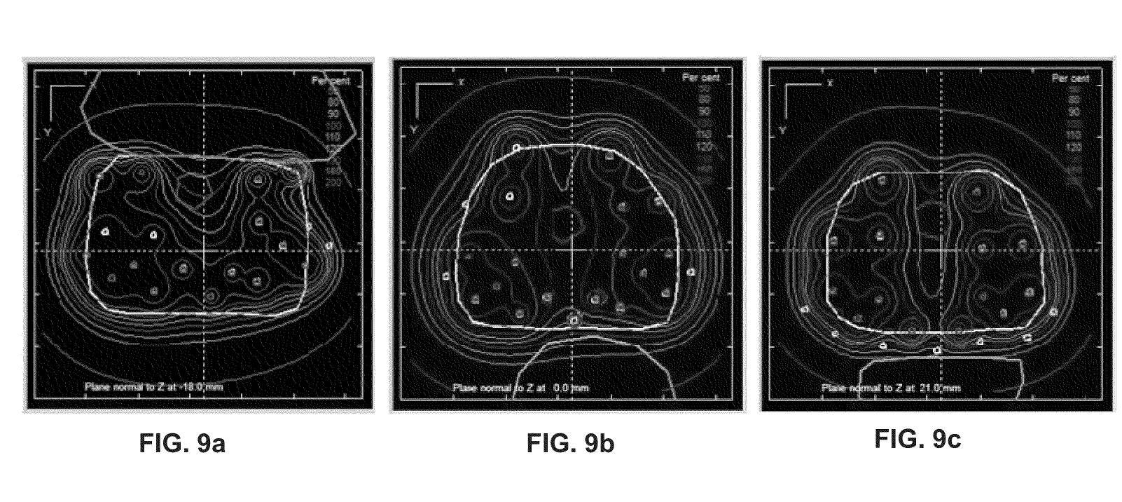

FIG. 8 illustrates exemplary graphs of the Cumulative DVHs of organs (e.g., “implant,” “target anatomical portion,” “blaas” (e.g., bladder), “rectum,” and “urethra”), including the volumes in these organs receiving a given dose. FIG. 8 also illustrates an exemplary graph of the Natural DVH of the implanted volume. The two prescription dose values (PD: Clinical Prescription dose, and NPD: Natural Prescription dose) are indicated in the combined graphs of the CDVH of the implanted volume and of the target anatomical portion and in the NDVH of the implanted volume.

The generation of the Cumulative DVH and Natural DVH graphs makes it possible for treatment planning system to give software-generated warnings to the user when the implant is not optimal and to offer suggestions as to how to adjust the treatment plan, based on the five disclosed indices. FIG. 8 illustrates an exemplary software-generated set of DVH parameters (labeled “Report DVH Parameters”), on which these warnings can be based, according to this disclosed embodiment. For example, in FIG. 8, therapy planning module 12a may provide a display to the user with an alert that the implant is not optimal, based on the values of the five disclosed indices (labeled “Essential parameters”). The treatment planning system may also provide, in the display, target coverage information, such as dosage and volume. As a result, the user can view the information in the display and determine whether the dose distribution of the implant is optimal.

The treatment planning system may also generate dose-volume tables of the implanted volume, the target anatomical portion, and the volumes of the organs at risk. Dose-volume tables are lists of volumes encompassed by isodose surfaces of decreasing dose values. The information from these tables is based on the Cumulative DVHs. The Report DVH Parameters presents a table of each Region of Interest (ROI) with the volumes encompassed by an range of dose values. For example, a physician may be interested in the highest dose received by 2 cm³ volume anywhere inside the rectum. In the table below, the value of 75% PD is found for 2.01 cm³. Also demonstrated is the highest dose for 0.1 cm³. From the table follows a value around 94% PD.

In order to use the IIP tool, the system may generate a real-time display of a dose-volume histogram (DVH), so that the user can evaluate the dose distribution in real-time. FIG. 8 illustrates an exemplary dose volume histogram of an EGO and IIP plan of a prostate implant. As illustrated, the Coverage Outside (“CO”) index is much smaller than 1.0, indicated by the implant red curve lying above the target curve in the Cumulative DVH. This indicates that a significant amount of healthy tissue outside the CTV is receiving a high dose of radiation, which is not optimal. Thus, the user may wish to change the dose distribution based on viewing these DVHs.

Thus, Cumulative DVHs of organs at risk give information on the amount of the organ's volume that is overdosed. It may not be sufficient, however, to state that the volume receives a dose higher than a given level. As described previously, it makes a clinical difference whether that volume consists of a single contiguous volume or of the sum of a set of small discontiguous subvolumes.

To obtain real-time display of the DVHs, the present disclosure aims to use randomly placed dose points to construct the DVHs. This set of randomly placed dose points may be used to determine the volumes given in FIG. 7a and FIG. 8. These volumes may or may not be contiguous. The use of randomly placed dose points inside the target volume and organs at risk to determine the DVH parameters and the volume-dose tables may be much more efficient than the use of an equidistant Cartesian grid.

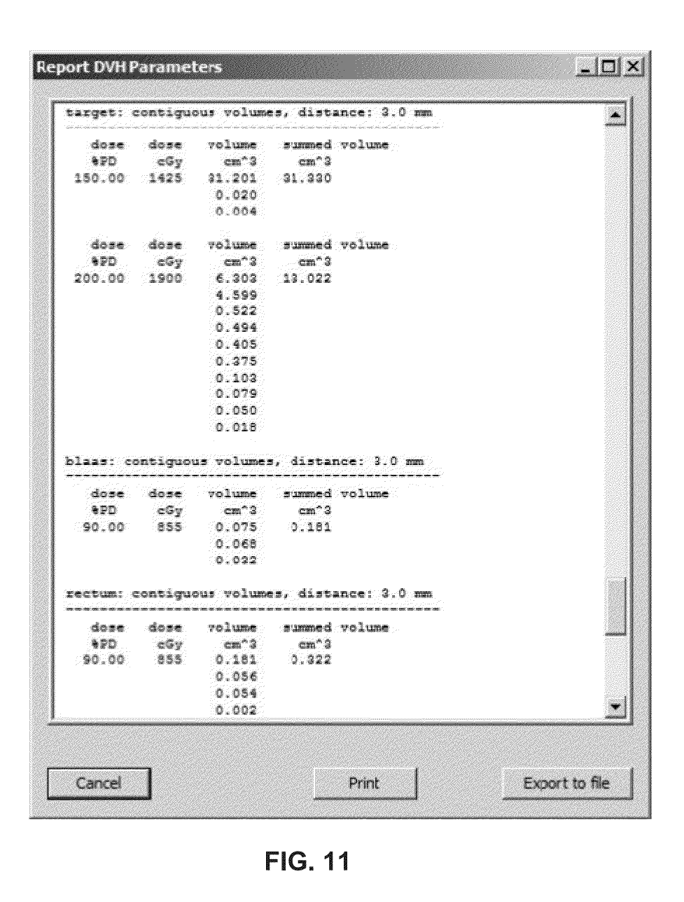

FIG. 10 discloses an exemplary process, according to the disclosed embodiments, for using randomly placed dose points to determine the contiguous volumes within regions of interest (ROIs), such as the clinical target volume or organs at risk. As shown in FIG. 10, the process may include selecting a first point within the ROI that has a dose higher than a set dose value (step 1002). Such a set dose value for, e.g., the rectum could be 90% of the prescription dose PD. The process may include repeatedly adding an outer layer of points (to the first point selected), where all the points in the outer layer have doses higher than the set dose value (steps 1004 and 1006), which grows the contiguous volume. As the points are randomly placed, a point may be added to the contiguous volume being constructed if its distance to any of the points in the outer layer (with a dose higher than the set value) is less than a set value, typically 3 mm. In this way, the process may grow a contiguous volume composed of outer layers of points around the first point. Further, the process may include continually adding outer layers of points until no such points can be found that have a dose higher than the set dose value (step 1006). Then, when all points belonging to this contiguous volume are found, the volume may be calculated by multiplying the number of points found with the voxel size. With randomly placed points, the voxel size of a point may be defined as the volume of the ROI divided by the total number of random points inside the ROI. The treatment planning system then displays a visual representation of the contiguous volumes by displaying the randomly placed points inside the region of interest with a dose greater than or equal to the set dose (step 1010).

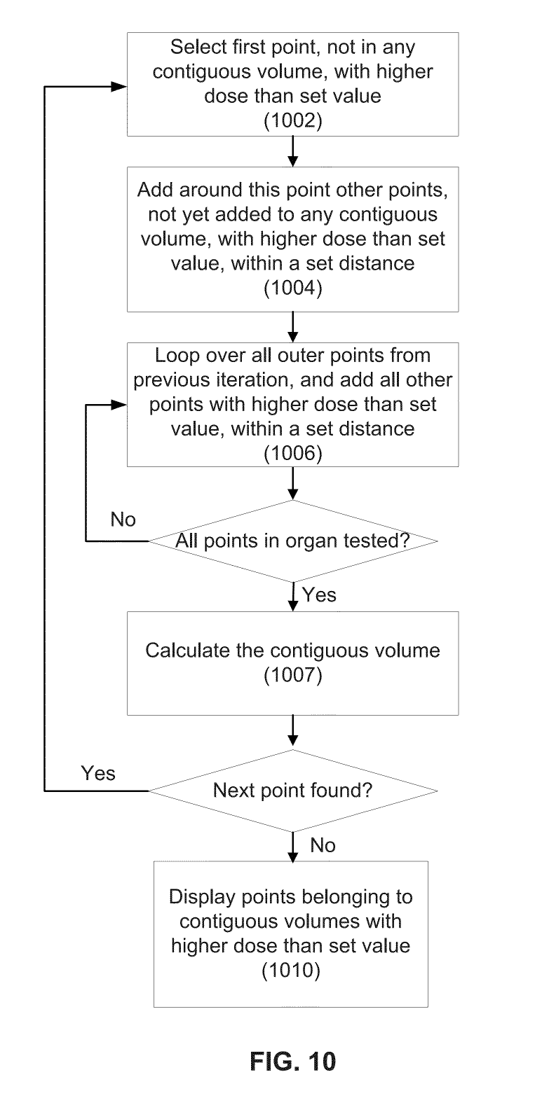

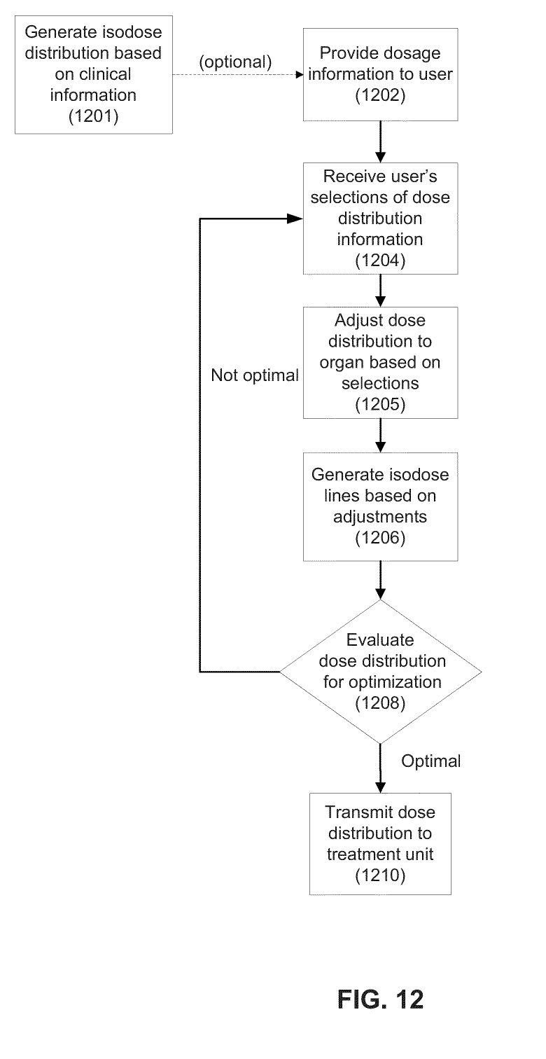

The process may also include generating a report of the contiguous volumes (step 1010). FIG. 11 illustrates an exemplary report of contiguous volumes for given dose limits for several ROIs: the target, the bladder, and the rectum. FIG. 11 illustrates the two large volumes in the target anatomical portion having doses of at least 200% PD—6.3 and 4.6 cm³—seen on the left side of FIG. 9a and on the right size of FIG. 9b. Both volumes can also be seen on the left and right side of FIG. 9c.

The foregoing description has been presented for purposes of illustration. It is not exhaustive and is not limiting to the precise forms or embodiments disclosed. Modifications and adaptations will be apparent to those skilled in the art from consideration of the specification and practice of the disclosed embodiments. For example, systems and methods consistent with the disclosed embodiments may be implemented as a combination of hardware and software or in hardware alone. Examples of hardware include computing or processing systems, including personal computers, laptops, mainframes, micro-processors and the like. Additionally, although aspects are described for being stored in memory, one skilled in the art will appreciate that these aspects can also be stored on other types of computer-readable media, such as secondary storage devices, for example, hard disks, floppy disks, or CD-ROM, or other forms of RAM or ROM.

Programmable instructions, including computer programs, based on the written description and disclosed embodiments are within the skill of an experienced developer. The various programs or program modules can be created using any of the techniques known to one skilled in the art or can be designed in connection with existing software. For example, program sections or program modules can be designed in or by means of C#, Java, C++, HTML, XML, or HTML with included Java applets. One or more of such software sections or modules can be integrated into a computer system or existing e-mail or browser software.

Moreover, while illustrative embodiments have been described herein, the scope of thereof includes any and all embodiments having equivalent elements, modifications, omissions, combinations (e.g., of aspects across various embodiments), adaptations and/or alterations as would be appreciated by those in the art based on the present disclosure. For example, the number and orientation of components shown in the exemplary systems may be modified. Further, with respect to the exemplary methods illustrated in the attached drawings, the order and sequence of steps may be modified, and steps may be added or deleted.

The claims are to be interpreted broadly based on the language employed in the claims and not limited to examples described in the present specification, which examples are to be construed as non-exclusive. Further, the steps of the disclosed methods may be modified in any manner, including by reordering steps and/or inserting or deleting steps.

It is intended, therefore, that the specification and examples be considered as exemplary only. Additional embodiments are within the purview of the present disclosure.

1. A radiation therapy treatment planning method for adjusting a dose distribution of a target anatomical portion having an implanted volume, the method comprising:
displaying an image of the implanted volume and a set of catheters displayed as implanted in the anatomical portion;
determining to adjust the dose distribution to the implanted volume that is configured to be delivered via the set of catheters;
receiving, at a processor, a selection of a dose distribution parameter that affects the strength of the dose distribution to the implanted volume;
adjusting, based on the received dose distribution parameter, a dwell weight of the radiation dose at each dose position in the set of catheters, wherein the adjusted dwell weight adjusts the dose distribution relative to a center region of the implanted volume such that the distribution moves to or from an outer circumference of the target anatomical portion.

2. The method of claim 1, further comprising:
generating, by the processor, a displayable second image of the implanted volume that reflects the adjusted dose distribution.

3. The method of claim 1, wherein the displaying further comprises displaying a geometrically adjusted dose distribution of the implanted volume.

4. The method of claim 3, wherein the determining further comprises determining whether the geometrically adjusted dose distribution of the implanted volume needs further adjustment.

5. The method of claim 1, wherein the determining further comprises determining that the dose distribution of the implanted volume is not homogenous.

6. The method of claim 1, further comprising:
receiving, at a processor, a selection of a volume range of the dose distribution reflecting a distance around each dose position.

7. A radiation therapy treatment planning method for adjusting a dose distribution of a target anatomical portion and an organ at risk, the target anatomical portion being proximate to the organ at risk, the dose distribution including a set of dose points randomly displaced throughout the target anatomical portion, the method comprising:
displaying a first image of the target anatomical portion, the first image including the organ at risk and a set of catheters;
determining that the dose distribution affecting the organ at risk needs further adjustment;
receiving, at a processor, a selection of a property of the dose distribution;
iteratively adjusting, by the processor, based on the received selection, the dose at each point in the set of points until a predefined characteristic of the dose distribution has been achieved; and
generating, by the processor, a second image of the target anatomical portion and the organ at risk.

8. The method of claim 7, wherein the property includes a dose limit to the organ at risk.

9. The method of claim 8, wherein the property includes at least one of an upper limit or a lower limit of the dose limit to the organ at risk.

10. The method of claim 8, wherein the property includes a percentage of the dose to the organ at risk.

11. A radiation therapy treatment planning method for determining contiguous volumes within a region of interest of a target anatomical portion, wherein a contiguous volume reflects a volume of the anatomical portion having a radiation dose higher than a set radiation dose value, the method comprising:
randomly selecting a first point within a 3-dimensional representation of the target anatomical portion, the first point associated with a higher radiation dose than the set radiation dose value;
iteratively adding a set of surrounding layers of points to the first point selected, each layer of points being in a layer around the previous surrounding layer of points and having higher radiation doses than the set radiation dose value; and
determining that no points around the outermost surrounding layer to the first point have higher radiation doses than the set radiation dose value; and
determining, based on the set of points in the set of surrounding layers, a first contiguous volume within the target anatomical portion, wherein the first contiguous volume has a radiation dose higher than the set radiation dose value.

12. The method according to claim 11, further comprising:
selecting a second point within a three-dimensional representation of the region of interest, the second point having a higher radiation dose than the set radiation dose value and being outside the first contiguous volume;
iteratively adding a set of surrounding layers of points to the selected second point, each layer of points being in a layer around the previous surrounding layer of points and having higher radiation doses than the set radiation dose value; and
determining that no points in the outermost surrounding layer to the second point have higher radiation doses than the set radiation dose value;
determining, based on the determined set of points in the set of surrounding layers of the second point, a second contiguous volume within the target anatomical portion, wherein the second contiguous volume has a radiation dose higher than the set radiation dose value; and
determining a summed volume of the first and second contiguous volumes found.

13. The method according to claim 11, further comprising:
generating a report of the contiguous volumes found, wherein the report reflects at least the contiguous volumes found and the corresponding lower dose limits of the contiguous volumes.

14. The method according to claim 11, further comprising:
setting the lower dose limit value for contiguous volumes for the target anatomical portion.

15. The method according to claim 14, wherein the lower dose limit value is set to a percentage of a prescription dose.

16. The method according to claim 11, further comprising:
setting the dose value for the contiguous volumes within an organ at risk, the organ at risk being proximate to the target anatomical portion.

17. The method according to claim 16, wherein the set dose value for the contiguous volumes is lower than a clinically prescribed value.

18. A computer-implemented method of radiation therapy treatment planning for determining at least the contiguous volumes inside the target anatomical portion, having a radiation dose higher than a set radiation dose value, the method comprising:
randomly selecting a first point within a three-dimensional representation of the target anatomical portion, the first point associated with a higher radiation dose than the set radiation dose value;
iteratively adding a set of surrounding layers of points to the selected first point, each layer of points being in a layer around the previous surrounding layer of points and having higher radiation doses than the set radiation dose value; and
determining that no points around the outermost surrounding layer to the first point have higher radiation doses than the set radiation dose value; and
determining, based on the set of points in the set of surrounding layers, a first contiguous volume within the target anatomical portion, wherein the first contiguous volume has a radiation dose higher than the set radiation dose value.

19. The method according to claim 18, further comprising:
selecting a second point within a three-Dimensional representation of the region of interest, the second point having a higher radiation dose than the set radiation dose value and being outside the first contiguous volume;
iteratively adding a set of surrounding layers of points to the second point selected, each layer of points being in a layer around the previous surrounding layer of points and having higher radiation doses than the set radiation dose value; and
determining that no points in the outermost surrounding layer to the second point have higher radiation doses than the set radiation dose value;
determining, based on the determined set of points in the set of surrounding layers of the second point, a second contiguous volume within the target anatomical portion, wherein the second contiguous volume has a radiation dose higher than the set radiation dose value; and
determining a summed volume of the first and second contiguous volumes found.
